(19) United States
(12) Patent Application Publication
(10) Pub. No.: US 2007/0123966 A1
(43) Pub. Date: May 31, 2007

(54) Pacemaker/defibrillator lead system

(76) Inventor: Daniel Rieders, Mount Dora, FL (US)

Correspondence Address: JEFFER, MANGELS, BUTLER & MARMARO, LLP, 1900 AVENUE OF THE STARS, 7TH FLOOR, LOS ANGELES, CA, 90067, US

(21) Appl. No.: 11/440,408

(22) Filed: May 23, 2006

Related U.S. Application Data
(60) Provisional application No. 60/684,100, filed on May 24, 2005.

(51) Int. Cl. A61N 1/00

(52) U.S. Cl. 607/122

(57) ABSTRACT

A lead system including a lead body having a distal end and a proximal end; a distal electrode comprising a conductive guide projecting from the distal end of the lead; and,a connector coupling the conductive guide to the lead body.

### FIELD OF THE INVENTION

The present invention relates generally to medical devices, and more particularly, to a pacemaker/ defibrillator lead system.

### BACKGROUND OF THE INVENTION

A natural pacemaker governs the beating of a heart. This natural pacemaker consists of a cluster of specialized cells constituting the sinoatrial (S-A) node, as illustrated in FIG. 10. Located in the right atrium of the heart, these cells cause the repeated contraction and relaxation of the heart by sending electrical signals along specific heart conduction pathways and at regular time intervals. Under normal conditions, the left and right ventricles contract fractions of a second after they have been filled with blood from an atrial contraction. The timing of this sequence of events is crucial for proper beating of the heart and is known as atrio-ventricular synchrony (A-V synchrony). When the heart deviates from this synchrony, a cardiac arrhythmia, or abnormal beating of the heart, results. An abnormally fast heart rhythm is called tachycardia, while an abnormally slow beating rhythm is known as bradycardia. In tachycardia, electrical signals deviate from the correct path along the heart muscle and cause the heart to beat exceptionally fast, and even quiver, instead of contracting. Patients with bradycardia also may experience changes in A-V synchrony, except this time, the S-A node does not send signals frequently enough for the heart to contract normally. A slow heartbeat may also result from delays or blockage of electrical signals in the heart.

The specialized cardiac conduction can also become diseased, causing the ventricles to fall out of synchrony. Specifically, the actual manner in which the ventricles contract fall out of synchrony. This dis-synchrony causes further cardiac decompensation, which becoming a vicious cycle of decompensation and heart failure.

Since its inception, cardiac pacing has continued to evolve, both from advances in technology as well as usage. For example, a novel pacing technique referred to as cardiac resynchronization therapy (CRT), also known as biventricular pacing, has been demonstrated to be beneficial to those inflicted with congestive heart failure (HF). There is strong evidence that this therapy favorably improves the patient survival history of this severe illness. By resynchronizing all of the four heart chambers, and specifically resynchronizing the left ventricular (the major pumping chamber of the heart), dramatic improvement in patients' clinical status are commonly seen.

Heart disease is a leading cause of death. It is estimated that there are nearly 5,000,000 HF patients currently in the United States, and a largely increasing population in the years to come is expected. Further, there are about 1,000,000 acute hospitalizations annually in the United States for HF, resulting in a profound financial burden to our healthcare system. CRT reduces rehospitalization for HF and improves mortality.

Currently, the lead that makes CRT possible is implanted transvenously (via the venous system) like other pacemaker or defibrillator leads (a defibrillator lead is generally implanted concurrently with CRT). However, there are significant procedural factors that make the implanting of the lead necessary for CRT difficult. Once access is obtained to the right heart, the coronary sinus is cannulated. Next, pictures are taken to demonstrate the coronary sinus and its tributaries. Then a guide wire is passed into a tributary and the lead is passed over the guide wire to different areas. The guide wire is used in conjunction with the lead for the purpose of guiding the lead to a target site. Once an optimal site is obtained, the apparatus used to deploy the lead, including the guide wire, is removed. There are many variations to this procedure because there are many patients with unique anatomical variations. Some of the greatest difficulties include:

Passing a wire to an adequate vessel.

Optimal site

Optimal pacing parameters.

Lead dislodgment.

During the lead implanting procedure, many measurements are obtained to identify an adequate pacing site. It is common practice to leave the guide wire advanced out a certain distance past the lead body while these measurements are being taken. The lead body can then be passed back and forth so that different locations along one or more tributaries can be tested. It is commonly recognized that falsely excellent parameters are noted while the wire is out past the body of the lead. Once the wire is withdrawn into the body of the lead, however, the results can deteriorate significantly-many times changing parameters from being outstanding to being unacceptable.

For example, it is commonly recognized that signals detected when the guide wire is extended a particular distance beyond the lead body during the process of testing the lead location often may be falsely excellent. Thus, once a lead location has been tested as a suitable lead location (i.e., a site that returns a desirable measurement), the guide wire is removed and the process of placing the lead should be complete. However, it has been discovered that, often times, once the guide wire is removed from the lead and the lead is again retested (to verify retention of the desired measurements), the resulting measurements are not at the same level as those that were previously indicated. In fact, once the guide wire is removed, there is often a noticeable decrease in the measurements. Also, many times a guide wire can be passed to a desired area, but the lead cannot be maneuvered there.

Accordingly, there is a need to overcome the issues noted above.

### SUMMARY OF THE PREFERRED EMBODIMENTS

In an effort to address the beneficial impact that the guide wire has when used with the lead in providing desired lead performance, an lead system was devised that comprises a lead having a conductive wire that extends a desired distance therefrom, and that is permanently retained in the extended position, i.e., is not extracted or otherwise removed once the lead is implanted, for the purpose of serving as a distal electrode.

What is described herein is a pacemaker/defibrillator lead comprising a distal electrode or conductor extending a distance from the lead generally, and according to the three following embodiments:

1. Normal lead+guide wire: In a first preferred embodiment, the guide wire is used in conjunction with a standard lead (having both a proximal node and distal node), wherein the guide wire projects a distance from the lead end and acts as an extension of the distal node.

2. Modified lead (no distal node)+guide wire: In a second preferred embodiment, the lead does not have a distal node, and the guide wire again projects a distance from the lead end and serves as the distal node.

3. Modified lead (no distal/proximal node)+guide wire: In a third preferred embodiment, the lead has neither a distal nor proximal node, the guide wire projects a distance from the end of the lead and is used as a distal node, and the metal casing of the pacemaker/defibrillator is used the proximal node.

The guide wire useful for each of these embodiments may be partially insulated such that only a portion of the end of the guide wire projecting from the end of the lead is electrically conductive. The conductive and non-conductive portion of the guide wire may of different lengths as called for by each particular application. Although the invention is applied to implementing a guide wire/lead for pacing, it may also be applied to similar types of applications where it is desirable to provide precise placement of electrodes.

Other features and advantages of the present invention will become apparent to those skilled in the art from the following detailed description. It is to be understood, however, that the detailed description of the various embodiments and specific examples, while indicating preferred and other embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

The invention may be more readily understood by referring to the accompanying drawings in which:

FIG. 1 is a cross-sectional view of a lead in a lead system configured in accordance with one preferred embodiment of the present invention;

FIG. 2 is a cross-sectional view of the lead system of FIG. 1, including a conductive guide inserted therein, configured in accordance with one preferred embodiment of the present invention;

FIG. 3 is a cross-sectional view of the lead system of FIG. 1, including the conductive guide as shown in FIG. 2 and a connector configured in accordance with one preferred embodiment of the present invention for coupling the lead system to a pacing device;

FIG. 4 is a cross-sectional view of a lead having an integrated connector for a lead system configured in accordance with a second preferred embodiment of the present invention;

FIG. 5 is a cross-sectional view of the lead system of FIG. 4 that includes a conductive guide inserted into the lead and secured in the integrated connector, as configured in accordance with the second preferred embodiment of the present invention;

FIG. 6 is a cross-sectional view of a connector that can be used to connect the various lead systems described herein, configured in accordance with one embodiment of the present invention;

FIG. 7 is a cross-sectional view of a lead system configured in accordance with a third preferred embodiment of the present invention where the lead system includes a lead having a distal node in addition to a conductive guide that is secured to the lead with a connector configured in accordance with one preferred embodiment of the present invention;

FIG. 8 is a cross-sectional view of a lead system configured in accordance with a fourth preferred embodiment of the present invention where the lead system includes a lead having a distal node in addition to an internal electrical coupling for a conductive guide secured to the lead with a connector configured in accordance with another preferred embodiment of the present invention;

FIG. 9 is a cross-sectional view of a lead system configured in accordance with a fifth preferred embodiment of the present invention where the lead system includes a conductive guide secured to a lead by a connector with a proximal node; and,

FIG. 10 is an interior view of a heart, illustrating the specialized cells in the sinoatrial node of the heart.

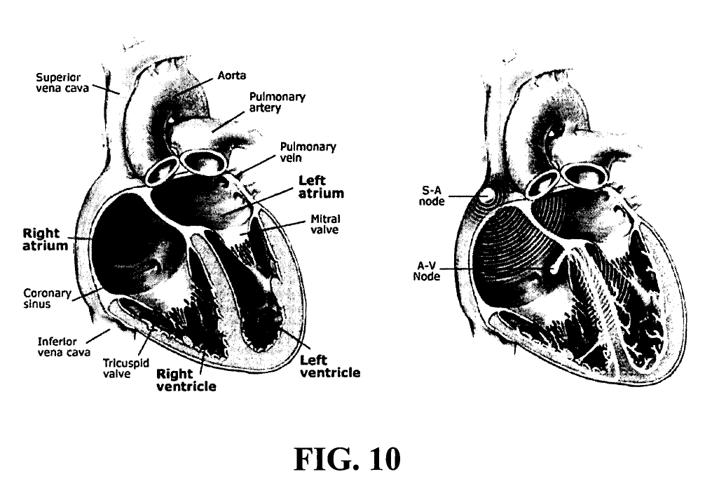

Like numerals refer to like parts throughout the several views of the drawings.

### DETAILED DESCRIPTION OF THE PREFERRED EMBODIMENTS

The essence of the invention is that the guide wire is insulated and the tip, or another portion, is the electrode that serves to sense and/or pace. This is like the patented pacemaker lead, but novel in that it is miniaturized so that the guide wire is actually the lead. The other part of the invention is the proximal end which attaches to the pulse generator or adapter.

The present invention is a lead system that includes a lead, an integrated conductive guide electrode, and a guide securing connector that couples the conductive guide to the lead. In one preferred embodiment, the inventive lead system includes a lead having a tip (distal) portion with a conductive guide that may be extended a desired distance therefrom, where the conductive guide is permanently retained in the extended position, i.e., is not extracted or otherwise moved with respect to the lead once the lead system is implanted, by the guide securing connector. In this configuration, the conductive guide serves the purpose of a distal node, which is a surface where electrical contact may be made with a patient to provide pacing or other electrical signals to/from a pacing device. For example, these conductive guides function to both provide “sense” signals to a device such as a pulse generator, and, when needed, to pace. The conductive guides may also provide electrical signaling for a device that implements other functions. The end of the lead on which the guide securing connector is attached, which is referred to as the proximal end, is the end attached to a pacemaking pulse generator.

The present invention may be implemented to create unipolar lead systems, which include leads that do not include a proximal electrode. In other embodiments, the lead system may also include additional nodes located either at the distal or proximal, or both, ends of the lead, such as those on bipolar leads that include a node on the lead body and another node on the distal tip. The conductive guide as well as any nodes on the lead are coupled to one or more electrodes on the guide securing connector with which the pacing device is later connected.

To facilitate interchangeability of pacing devices, industry standards specify certain features of lead and device connectors. For example, the standards organization named the International Organization for Standardization promulgates a standard for low-profile connectors for implantable pacemakers officially designated as ISO 5841.3:2000, but more commonly referred to as an “IS-1 connector.” In one embodiment, the present invention provides a connector that is compatible with any pacing device that can interface with an IS-1 connector.

Further, in one preferred embodiment of the present invention, the conductive guides described above may be partially insulated such that only a portion of the distal end (or another portion) of the conductive guides projecting from the end of the lead is electrically conductive. The conductive and non-conductive portions of the conductive guides may be of different lengths as required by a particular application. In addition, the conductive guides may be trimmed as necessary during the surgery, with the connectors and adapters described above having space to accept non-cleanly trimmed conductive guides but still being able to be inserted into a pacing device. Although the invention is applied to implementing a lead system for pacing, it may also be applied to other types of applications where it is desirable to provide precise placement of electrodes.

The inventive lead system will be described with respect to the three following preferred embodiments of the present invention:

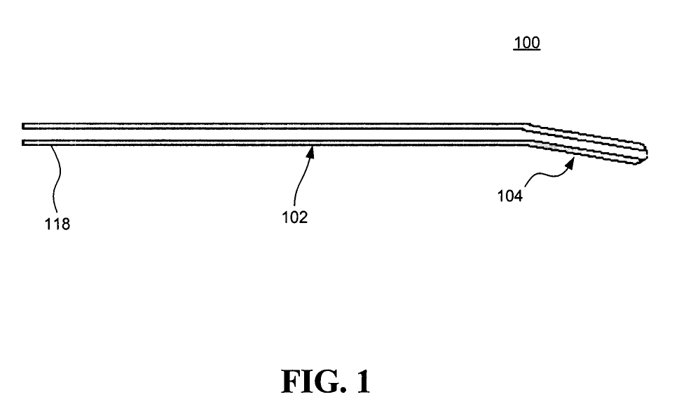

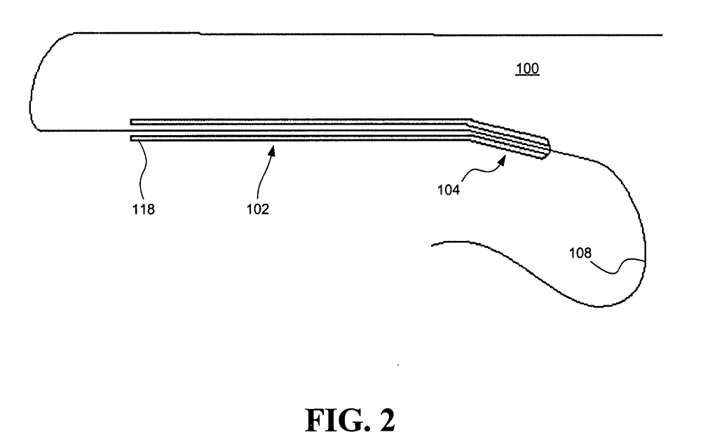

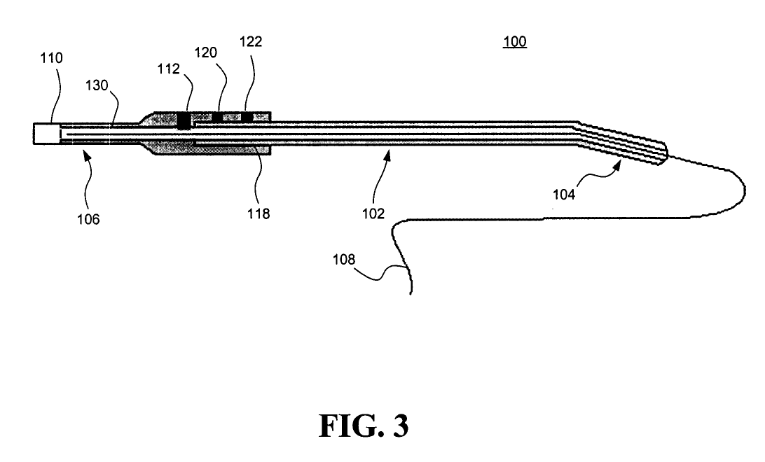

1. A lead system that includes a lead that does not include a distal node, where a conductive guide that projects a distance from the distal end of the lead is used as the distal electrode. This embodiment is illustrated by the lead system shown in the figures from FIG. 1 to FIG. 3.

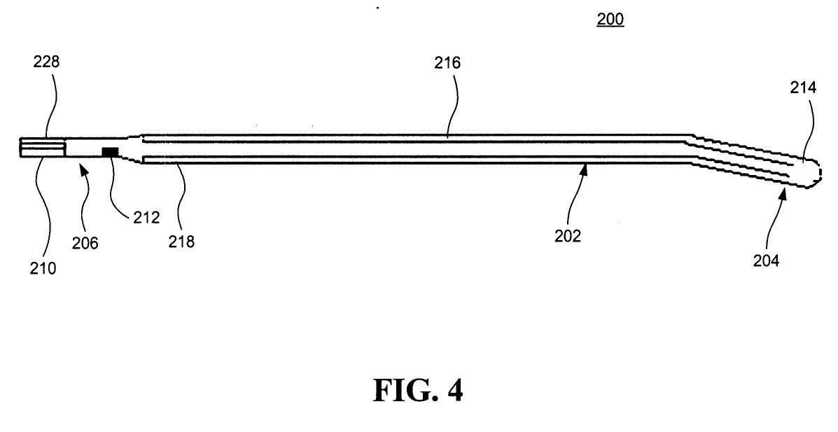

2. A lead system with a lead having a distal node, the functionality of which is enhanced by a conductive guide. Embodiments of this type of lead system are shown in the series of figures from FIG. 4 to FIG. 5 and FIG. 7 to FIG. 8.

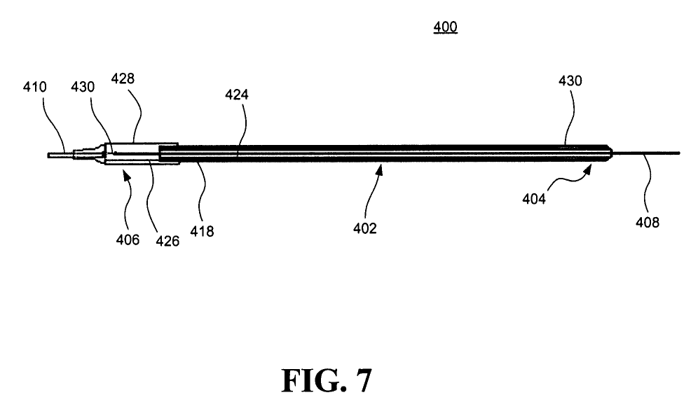

3. A lead system with a lead having both a distal and a proximal node, where a conductive guide may be used to enhance the functionality of the distal node as shown in FIG. 7 to FIG. 9.

The first and second embodiments described above may also not include a proximal end, in which case the metal housing of the pacing device is thus used as the proximal node. For example, the lead system as described in embodiment one would include a lead that has neither distal nor proximal nodes on the lead itself, and a conductive guide projects a distance from the distal end of the lead to be used as a distal node

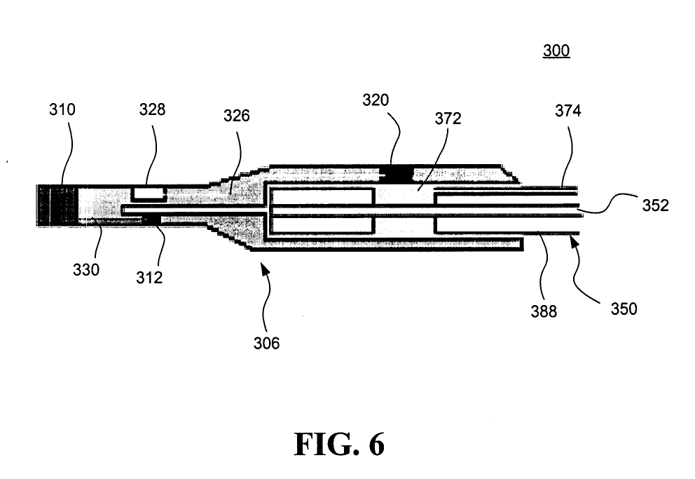

In addition to illustrating the various preferred embodiments of the lead systems of the present invention, the figures also illustrate the various preferred embodiments of connectors used to connect the lead system to a pacing device. In one preferred embodiment, the lead system includes an integrated connector. In another preferred embodiment, the system may use a separate connector, such as that shown in FIG. 6 and described in further detail below, that may be used to implement a lead system that provides the desired functionality of the inventive lead systems described herein, use existing leads and guide wires to. It should be noted that the use of the word “system” in the term “lead system” should not be read in a limiting fashion to require the specific features of the exemplary connectors used.

FIG. 1 to FIG. 3 illustrate a lead system 100 with a lead 102 having a distal end 104 and a proximal end 118, and where, in a deployed state, a conductive guide 108 is used as a distal node of the lead 102. In one embodiment, the installation of the lead system 100, the conductive guide 108 is used as an electrode to probe and locate a suitable target site, with the lead 102 slipped over the conductive guide 108. In another embodiment, the lead is not used if the guide wire is insulated and there is no need for more than one node during the site acquisition process. Once a suitable target site is located through the use of conductive guide 108, any excess length of conductive guide 108 is trimmed and the lead 102 is secured to the conductive guide 108 with a connector 106. In one preferred embodiment, the connector 106 includes a plurality of fasteners 120 and 122 used to secure the connector 106 to lead 02. The connector 106 also includes a screw 112 that is used to both secure the deployed position of the conductive guide 108 and electrically couple the conductive guide 108 to an internal wire 130 that is coupled to a distal electrode 110. In one preferred embodiment, the conductive guide 108 is insulated along its length except for a predetermined portion that is to be used as the distal node. In another embodiment, the conductive guide may be non-insulated along its entire length.

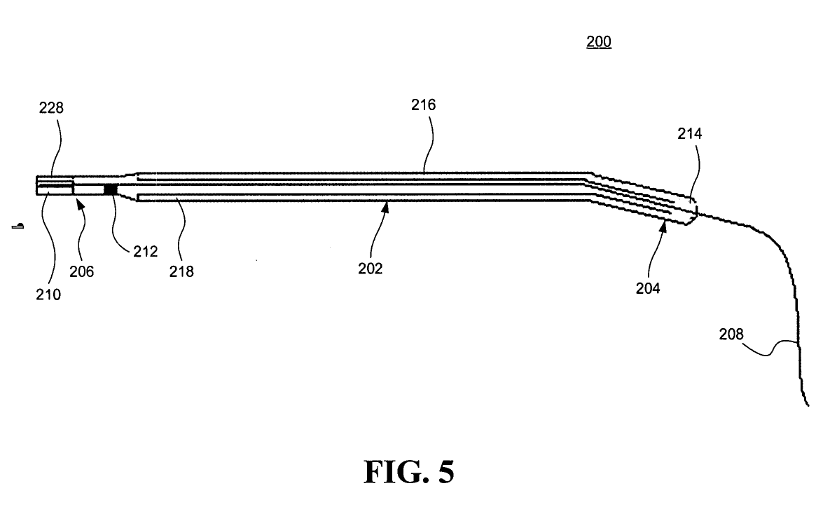

FIG. 4 and FIG. 5 illustrate a lead system 200 that includes a unipolar lead 202 with a distal node 214 at a distal end 204 and an integrated connector 206 on a proximal end 218. A fastener 212 secures the conductive guide 2008 at the distance at which it is deployed from lead 202 and also electrically couples the conductive guide 208 to an electrode 210. The distal node 214 is electrically coupled to a second electrode 228 through the use of a wire 216 that runs from the distal node along the length of lead 202.

FIG. 6 illustrates a connector 306 configured in accordance with one embodiment of the present invention that may be utilized with a prior art guide wire 352 and a prior art lead 350. The connector 306 is secured to a proximal end 388 of the lead 350 using a fastener 320. Fastener 320 simultaneously secures the lead 350 while electrically coupling a proximal wire 374, coming from a distal node (not shown) in lead 350, to a proximal electrode 328. As illustrated, the guide wire 352 is secured in the connector 306 by a fastener 312 that, similar to fastener 320, also provides the electrical coupling of the guide wire 352 to a distal electrode 310 through a distal wire 330.

During installation, the guide wire 352 would be used to locate a suitable target site using the standard methods as discussed previously, with the lead 350 being slipped over the guide wire 352. Once the target site is acquired, the proximal end of the guide wire, the lead 350, and any insulation will be trimmed so any excess length is removed and the connector 306 attached to the guide wire 312 and the lead 350 with fastener 312 and fastener 320, respectively. Thus, as described, the connector 306 will allow the use of prior art guide wires and leads. In addition, the guide wire 352 may optionally be secured by a fixation device (not shown) located at the distal end of either the guide wire 352 or lead 350 so that the likelihood of dislodgement of the guide wire 352 will be reduced.

FIG. 7 illustrates a lead system 400 where, in one preferred embodiment, a conductive guide 408 is used in conjunction with a lead 402 having both a proximal node 428 and a distal node 430 displaced on a proximal end 418 and a distal end 404, respectively, of the lead 402. Similar to the lead system 100 as shown in FIG. 1 to FIG. 3, in operation the conductive guide 408 is first inserted into the patient, followed by the lead 402. Once a target site providing a desired measurement has been located, a connector 406 is coupled to the conductive guide 408 and the lead 402 so as to affix the conductive guide 408 to the lead 402 and provide electrical connection to the lead system 400. The connector 406 will also hold the conductive guide 408 in the extended position. The connector 406 may optionally include additional electrical couplings 426 and 430 to better electrically connect the conductive guide 408 and the lead 402.

The conductive guide 408 may be used in two configurations, depending on the configuration of the electrical coupling of the guide wire 408 to the pacing device. In one preferred embodiment of the present invention, in the first configuration the conductive guide 408 projects a distance from the distal end 404 and acts as an electrical extension of the distal node 430. This configuration may also provide redundancy as there are now two contacts to the surface of the vessel, one being the distal node 430 and another being the conductive guide 408. In the second configuration, the conductive guide 408 acts as another node, separate from the distal node 430 of the lead 402.

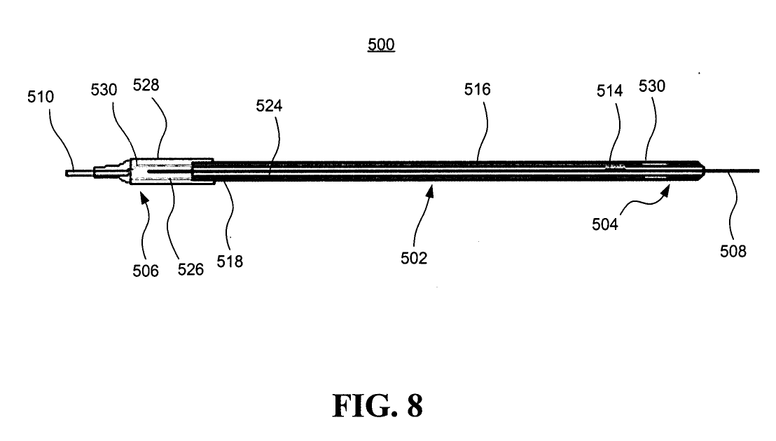

FIG. 8 illustrates another preferred embodiment of the present invention, where a lead system 500 includes a lead 502 with an internal connector 514 that electrically connects to a conductive guide 508 to internally connect the conductive guide 508 to a distal electrode 510 of the lead 502. This design, as well as the other designs described herein, provides a cavity internal to the lead 102 for adapting to the imperfection resulting from any necessary trimming of the conductive guide 508. A distal lead wire 524 couples a distal node 530 to a distal electrode 526.

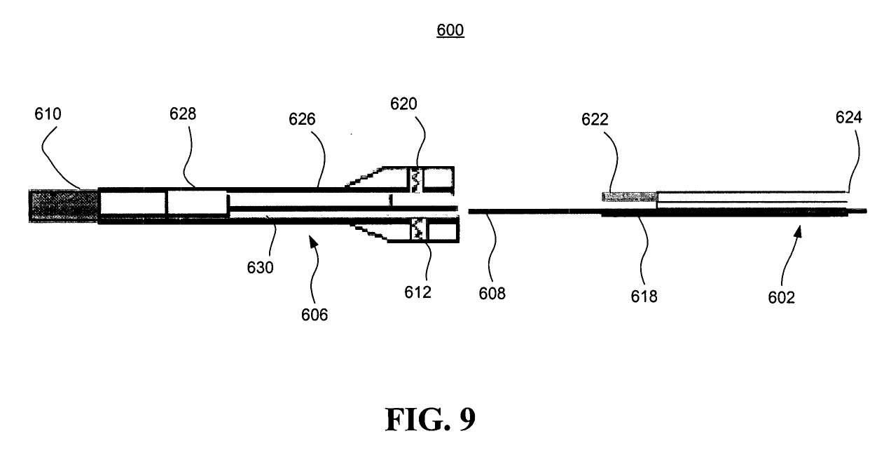

FIG. 9 is another embodiment of a lead system 600 having a connector 606 with fasteners 612 and 620 used to secure a conductive guide 608 and a proximal lead electrode 622, respectively, as well as electrically connect a proximal node wire 624 and the conductive guide 608 to a proximal electrode wire 626 and distal electrode wire 630, respectively. The proximal electrode wire 626 is coupled to a proximal electrode 628 on the connector 606. Similarly, the distal node wire 630 is coupled to a distal electrode 610 on the connector 606. In one embodiment, the space in which the conductive guide 608 fits is used for accommodating inaccuracies in the trimming of the conductive guide. Also, conductive guides of various fixed lengths may be used.

It should be understood that a lead system of present invention, exemplified by the lead systems embodied in the figures and descriptions presented above, may be used with various types of cardiac tissues, as categorized in two groups:

1. Ordinary myocardium (atrial and ventricular).

2. Specialized cardiac conduction system, which includes sinoatrial, or sinus node; anterior, middle and posterior intemodal tracts; atrioventricular (AV) node; His bundle; right and left bundle branches; anterior-superior and posterior-inferior divisions of the left bundle and the Purkinje network.

The embodiments described above are exemplary embodiments of the present invention. Those skilled in the art may now make numerous uses of, and departures from, the above-described embodiments without departing from the inventive concepts disclosed herein.

1. A lead system comprising:
a lead body having a distal end and a proximal end;
a distal electrode comprising a conductive guide projecting from the distal end of the lead; and,
a connector coupling the conductive guide to the lead body.

2. The lead system of claim 1, wherein the connector comprises a fastener configured to fixedly couple the connector to the lead body.

3. The lead system of claim 1, further comprising a distal node displaced on the lead body on the distal end.

4. The lead system of claim 3, wherein the connector comprises a fastener that is used to electrically couple the conductive guide to the distal node.

5. The lead system of claim 3, the lead body further comprising a conductor configured to electrically couple the distal node to the connector.

6. The lead system of claim 1, wherein the conductive guide comprises insulation along its length except for a predetermined portion.

7. The lead system of claim 1, further comprising a proximal node displaced on the proximal end of the lead body.

8. The lead system of claim 7, the lead body further comprising a conductor configured to electrically couple the proximal node to the connector.
